(19) United States
(12) Patent Application Publication
(10) Pub. No.: US 2008/0306423 A1
(43) Pub. Date: Dec. 11, 2008

(54) Combined ankle brace and soccer shin sleeve

(76) Inventor: Chris Gould, Mission Viejo, CA (US)

Correspondence Address: MICHAEL A. SHIPPEY, PH. D., LAW OFFICES OF KARLA SHIPPEY, 4848 LAKEVIEW AVENUE, SUITE E, YORBA LINDA, CA, 92886, US

(21) Appl. No.: 11/810,428

(22) Filed: Jun. 5, 2007

(51) Int. Cl. A61F 5/00; A41D 13/00

(52) U.S. Cl. 602/27; 2/22

(57) ABSTRACT

This invention is an ankle brace combination apparatus, designed for use in playing the game of soccer. The brace combination comprises a foot and ankle support unit having a single open resilient stirrup for the foot, ankle, and lower shin; a dual support strap structure operatively connected to said stirrup to extend over said foot portion and then to extend generally upwardly and rearwardly for retention to an anchor on the central support stirrup; and an optional pocket inside the upper portion of the central support stirrup for holding securely a shin guard.

### BACKGROUND

U.S. Pat. No. 7,014,621, to inventor Nelson on Mar. 21, 2006, describes an ankle brace comprising a base of flexible material shaped to wrap around the sides of a foot and ankle and underneath a portion of the foot, with a support strap for protecting the anterior talofibular ligament.

U.S. Pat. No. 6,447,621, to inventor Ritchie on Sep. 10, 2002, describes an ankle support and method for providing ankle support, which includes a stiff stirrup and ankle strap having an elongated, generally straight shin portion and terminating in an elongated, generally straight stirrup portion, the stirrup portion being at an obtuse angle to the shin portion.

U.S. Pat. No. 6,117,098, to inventors Weber et. al., dated Sep. 12, 2000, describes an ankle brace apparatus, including a foot and ankle holder having a foot portion, and having an upper ankle portion, and retention flaps connected to the holder and extending forwardly to overlap support strap structure at a retention zone.

U.S. Pat. No. 5,620,413, to inventors Deirmendjian et. al., dated Apr. 15, 1997, describes an ankle brace comprising an ankle anchor strap and first and second bracing straps to raise the heel of the wearer.

### BRIEF DESCRIPTION OF THE DRAWINGS

FIG. 1 is a side view of the preferred embodiment of the current invention.

FIG. 2 is a frontal view of the current invention, worn by a person, but as yet not fully assembled for use.

FIG. 3 is a frontal view of the current invention, again worn by a person, showing the attachment of the shorter strap.

FIG. 4 is a frontal view of the current invention, again worn by a person, showing the attachment of the longer strap.

FIG. 5 is a frontal view of the current invention, again worn by a person, now fully assembled for use.

FIG. 6 is a frontal view of the current invention, fully assembled, with a shin guard displayed for insertion.

FIG. 7 is a frontal view of the current invention, fully assembled, with a shin guard being inserted at the appropriate position in front of the shin of the player.

FIG. 8 is a frontal view of the current invention, fully assembled, with a shin guard fully inserted at the appropriate position.

The drawings include a description of a preferred embodiment of the current invention, and are incorporated into this specification for that purpose.

### DETAILED DESCRIPTION OF THE PREFERRED EMBODIMENTS

Detailed descriptions of the preferred embodiment are provided herein. It is to be understood, however, that the present invention may be embodied in various forms. Therefore, specific details disclosed herein are not to be interpreted as limiting, but rather as a basis for the claims and as representative basis for teaching one skilled in the art to employ the present invention in virtually any appropriately detailed system, structure or manner.

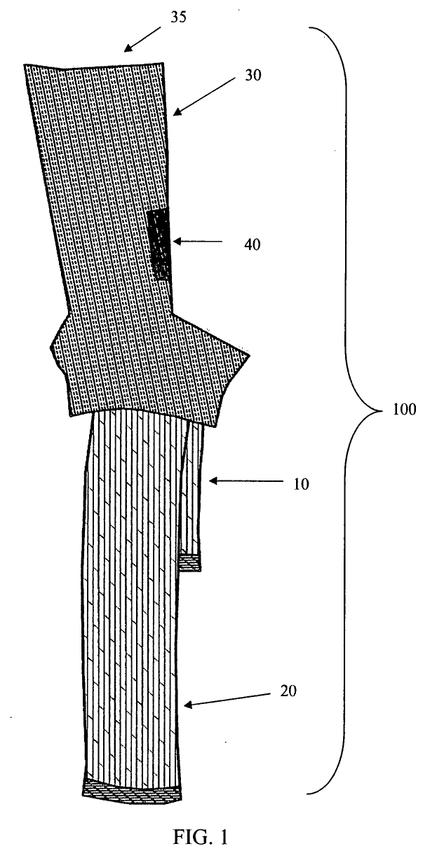

A preferred embodiment 100 of the current invention is portrayed in FIG. 1 in a side view. The brace includes a stirrup 30 with straps 10 and 20, of unequal length, hanging below. The stirrup has an anchor point 40 for removable attachment of the free ends of straps 10 and 20. Inside the open top of stirrup 30 is a pocket 35 which is designed to securely hold a shin guard. Stirrup 30 comprises a foot covering portion, and a shin covering portion that extends upward at least to the top of the shin, just below the bottom of the knee. Preferably stirrup 30 is comprised of washable cloth or semi-elastic fabric that is durable, and will withstand repeated use and hard impacts during repeated games of soccer.

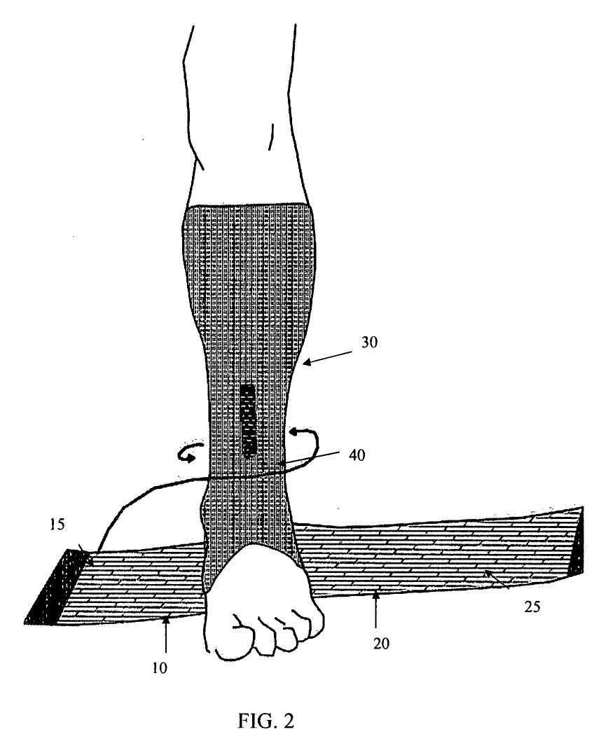

FIGS. 2-4 demonstrate the process of donning the brace or stirrup corresponding to the preferred embodiment of the current invention. In FIG. 2, the player has put the current invention onto his left foot and leg. The toes extend through the open toe of the foot portion. Straps 10 (the shorter) and 20 (longer) are extended flat onto the ground or floor.

To begin the process of assembling the brace, short strap 10 is pulled up and over the foot portion, and counter-clockwise around the back of the leg, ultimately terminating at the anchor point 40 of stirrup 30. Both anchor point 40 and free end 15 of strap 10 are comprised of hook and loop material. Both can removably attach to each other, or to the free end 25 of the other strap, 20.

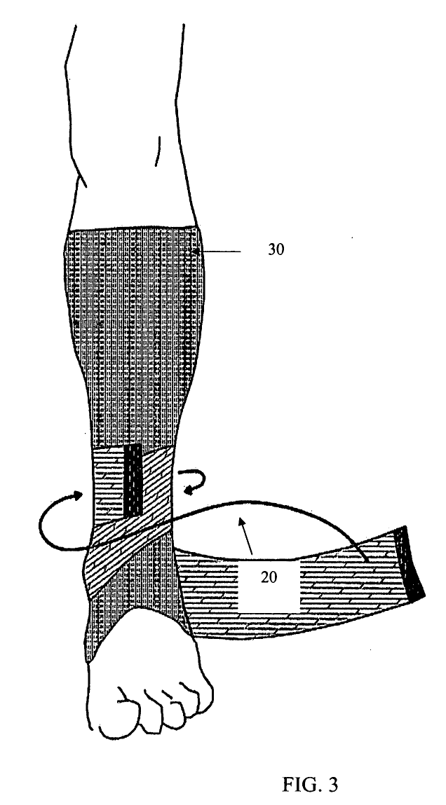

The process is continued in FIG. 3. Strap 20 now attached, strap 10 is moved in the opposite direction, clockwise over the front of the foot and around behind the leg, to again terminate in a removable connection to the anchor point 40 of stirrup 30.

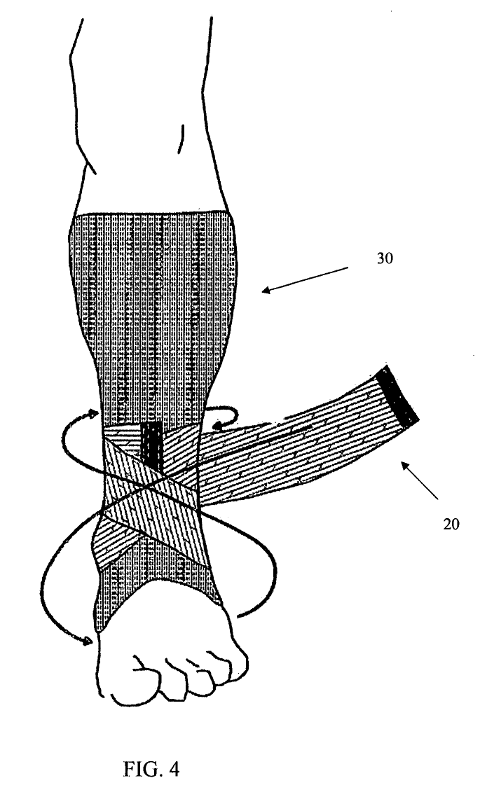

FIG. 4 shows the continuation of the clockwise move of the strap 20. As FIG. 4 demonstrates, it is possible by using longer straps to wrap one or both straps around the underside of the foot. This is preferable in certain situations, such as severe ankle sprains. However, it is not normally used by a person about to participate in a game of soccer.

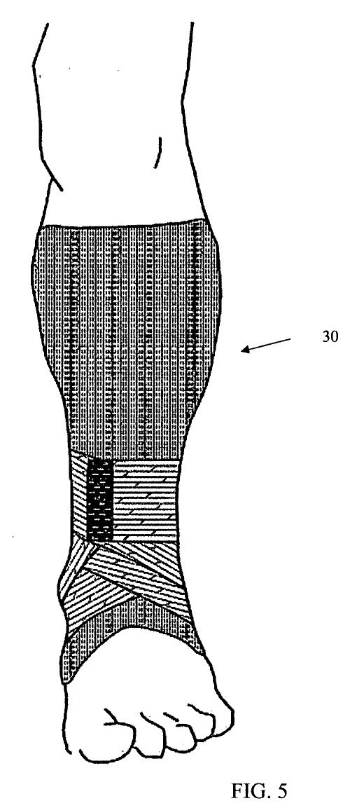

FIG. 5 shows the fully assembled brace of the current invention. A shin guard can now be placed inside stirrup 30. The shin covering portion of stirrup extends sufficiently high up the leg to completely cover the shin guard after the guard is inserted. The player then dons regular long socks and soccer shoes, and is properly equipped to play the game of soccer, while retaining protection for shin, lower leg, ankle and foot.

The short strap 10 of the current invention, when properly arrayed, is designed to protect the ankle from rolling inward and thereby straining ligaments in the foot, leg, and ankle. The longer strap 12 of the current invention, when properly arrayed, is designed to protect the ankle from rolling outward. It also protects against high ankle sprains.

The current invention also is designed to cooperate with the shin guard, to prevent it from sliding down out of position. Optionally, a pocket can be sewn into the inside of the shin portion of stirrup 30, in order to support a shin guard. However, testing has shown that a shin guard is adequately supported inside stirrup 30 without need of a pocket, as the straps 10, 12 prevent a shin guard from slipping down the leg of a player, even during play.

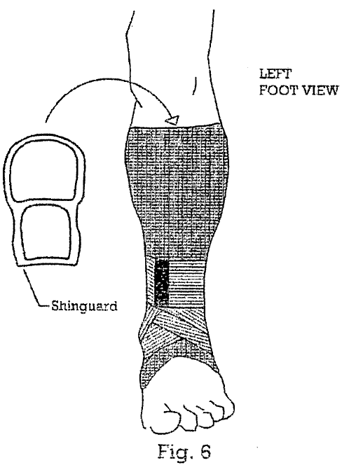

The mode of cooperatively using a shin guard with the current invention is shown in a progression of 3 figures, beginning with FIG. 6. In this figure, a shin guard is shown beside the current invention, assembled and worn on the leg of a player. An arrow shows where the shin guard is to be applied. It is inserted behind the upper front portion of stirrup 30 of the current invention.

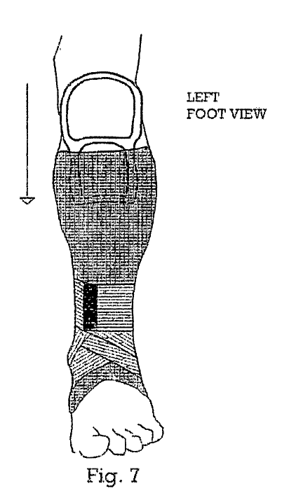

FIG. 7 shows the shin guard being inserted in the correct position. A shin guard is often worn inside the sock of a player. Using the current invention, the shin guard can be inserted inside a sock, in the traditional fashion. Alternatively, it can be worn outside the sock, between the sock and the shin portion of the current invention.

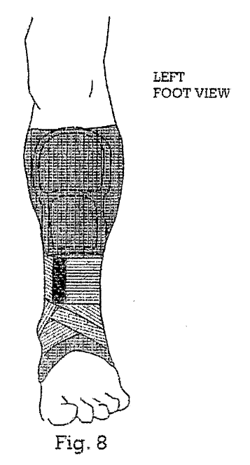

FIG. 8 displays a frontal view of the current invention, fully assembled, with a shin guard in place behind the front portion of stirrup 30. The shin guard is shown in dotted lines to indicate it is behind stirrup 30, and not exposed to view when fully inserted.

While the invention has been described in connection with a preferred embodiment or embodiments, it is not intended to limit the scope of the invention to the particular form set forth, but on the contrary, it is intended to cover such alternatives, modifications, and equivalents as may be included within the spirit and scope of the invention as defined by the appended claims.

I claim:

1. An ankle brace combination, the combination comprising
a foot and ankle stirrup having a foot portion, and having an upper ankle and shin portion with open top;
a plurality of support straps, each having a first end and a second end; each first end fixedly connected to said stirrup; the corresponding strap designed to extend under said foot portion and then to extend generally upwardly and rearwardly for removable retention to said stirrup at the second end thereof; and,
means for removable connection of the second end of each said support strap to said stirrup.

2. The combination of claim 1 wherein said support straps include a first strap and a second strap, the first end of said first strap being fixedly attached on one said of said foot portion of the stirrup, and the first end of said second strap being fixedly attached on the opposing side of said of said foot portion of the stirrup.

3. The combination of claim 2 wherein said first strap is twice as long as said second strap.

4. The combination of claim 3 wherein said first strap is fastened along the outer side of said foot portion, and said second strap is fastened along the inner side thereof.

5. The combination of claim 1 wherein said foot portion has an open toe portion.

6. The combination of claim 1 wherein said foot portion has an open heel portion.

7. The combination of claim 1, further comprising a hook-and-loop attachment anchor on the shin portion of said stirrup, and corresponding hook-and-loop attachment portions at the second ends of each said strap.

8. The combination of claim 1, further comprising a pocket at the front of the shin portion of said stirrup.

9. The combination of claim 8 wherein said pocket is designed to hold a shin guard.

10. A method for the attachment of the combination of claim 1 to a wearer; said method comprising:
placing the wearer's leg and foot into the stirrup;
wrapping the second strap upwardly over the front of the foot, then upwardly around the back and side of the ankle, then fastening the loose end at an anchor attachment point on the front of the shin portion of the stirrup; and
wrapping the first strap upwardly over the front of the foot, then upwardly around the back and side of the ankle, then underneath the foot, and back up fastening the loose end at an anchor attachment point on the front of the shin portion of the stirrup, thereby covering part of the second strap.

11. The method of claim 10, further including the step of inserting a shin guard into the inside between the upper stirrup and the leg.

12. The method of claim 10, further comprising said plurality of support straps including two support straps wrapped in opposite directions around said stirrup.

13. The combination of claim 1 wherein said straps are constructed of resilient material.

14. An ankle brace combination, the combination comprising
a foot and ankle stirrup having a foot portion, and having an upper ankle and shin portion with open top;
a shin guard, secured within the area at the front of the leg between shin and upper stirrup;
a pair of support straps, one longer than the other; each having a first end and a second end; each first end fixedly connected to said stirrup; the corresponding strap designed to extend under said foot portion and then to extend generally upwardly and rearwardly for removable retention to said stirrup at the second end thereof; and,
hook and loop means for removable connection of the second end of each said support strap to said stirrup.
